(19) United States
(12) Patent Application Publication
(10) Pub. No.: US 2015/0196420 A1
(43) Pub. Date: Jul. 16, 2015

(54) DEVICE FOR IMPROVING AIR FLOW THROUGH A NASAL CAVITY DURING PHYSICAL ACTIVITY SUCH AS SPROTING PURSUITS

(71) Applicant: ASAP BREATHEASSIST PTY LTD, Armadale, VIC (AU)

(72) Inventors: Nicholas Jon Ede, Victoria (AU); Michael Ralph Burgess Johnson, Victoria (AU)

(73) Assignee: ASAP BREATHEASSIST PTY LTD., Victoria (AU)

(21) Appl. No.: 14/417,451

(22) PCT Filed: Jan. 21, 2013
(86) PCT No.: PCT/AU2013/000043

§ 371 Date: Jan. 26, 2015

(30) Foreign Application Priority Data
Jul. 27, 2012 (AU) PCT/AU2012/000898

(51) Int. Cl. A61F 5/08; A61M 31/00

(52) U.S. Cl. CPC A61F 5/08; A61M 31/002

(57) ABSTRACT

There is disclosed herein a device for improving flow of air through the nasal cavity of wearer of the device, the device comprising a substantially U-shaped body forming a bridge for spanning the nostrils of a wearer when the device is in use; and a first body and a second body interconnected with opposite upright portions of the U-shaped body, each comprising a rib which extends outwardly from a portion of the U-shaped body distal to the bridge and ends in a section provided with padding which forms a relatively large surface area portion, wherein the first and second bodies are adjustable such that each relatively large surface area portion can be urged against a nostril wall, whereby resistance to flow of air is substantially reduced.

### FIELD OF THE INVENTION

The present invention relates to improved devices for facilitating respiration through the nostrils. In particular, the present invention relates to improved dilation devices which can be fitted to the nose to facilitate breathing. The invention has particular, though by no means exclusive, application during physical activity, in which increased air flow capacity is required.

### BACKGROUND OF THE INVENTION

During physical exercise, such as cycling, football, hockey or the like, it is common for a person to wear a resilient plaster applied over the bridge of the nose to expand the nostrils externally whereby to dilate the nostrils so that the person can gain an advantage from improved air flow through the nasal cavity. A problem associated with such a plaster is that the effect is limited by both the inherent resiliency of the plaster and its ability to exert a sufficient outward force to expand a nostril cavity and maintain it in a dilated condition. In addition, removal of the plaster can be painful and may cause tearing of skin.

Other devices for use in dilating the nostrils. One such device, disclosed in U.S. Pat. No. 3,710,799, comprises a pair of cage structures joined together by a flexible chain of interlocked links, the cages being slightly larger cross-sectionally than the nostrils and insertable therein so as to be held in place thereby. Fitting of this device can cause discomfort because each cage structure is generally larger than the corresponding orifice into which it is to be inserted. Conversely, if the cages are too small, they will not effect satisfactory dilation and/or may be dislodged during physical activity. Such a device may be useful while a wearer is asleep but impractical for use during physical exercise because the cage structures can obstruct air flow, which is greater during physical exertion.

Other devices for dilating the nostrils, such as that disclosed in U.S. Pat. No. 5,895,409, have a rigid structure and must be made in different sizes to suit differing nostril sizes and configurations. Also, such devices can be inadequate where the wearer has a deviated (curved) septum (the cartilage and bone that separates the nostrils). A deviated septum generally renders one nostril cavity different in size and shape from the other, whereby airflow through one side of the nose is obstructed.

Prior art devices of the type disclosed in U.S. Pat. No. 5,895,409, which comprises a symmetrical pair of dilators insertable within the nostril cavities, may suffer the drawback that if one dilator is sufficiently small to enter one nostril, the other dilator is too small to dilate the other nostril satisfactorily, or, conversely, if one dilator is sufficiently large to dilate one nostril, the other is too large to fit in the other nostril.

There is thus an ongoing need for improved devices which facilitate breathing through a person's nose, particularly during exercise, which are comfortable and can be universally fitted.

Also, breathing can be hindered by nasal congestion caused by allergic rhinitis, such as hay fever, or illness, such as a viral or bacterial infection (e.g. the common cold). It would be advantageous to be able, while dilating the nostrils, to treat congestion which hinders breathing.

### SUMMARY OF THE INVENTION

In accordance with a first aspect of the present invention, there is provided a device for improving flow of air through the nasal cavity of wearer of the device, the device comprising a substantially U-shaped body forming a bridge for spanning the nostrils of a wearer when the device is in use; and a first body and a second body interconnected with opposite upright portions of the U-shaped body, each comprising a rib which extends outwardly from a portion of the U-shaped body distal to the bridge and ends in a section provided with padding which forms a relatively large surface area portion, wherein the first and second bodies are adjustable such that each relatively large surface area portion can be urged against a nostril wall, whereby resistance to flow of air is substantially reduced.

In accordance with a second aspect of the present invention, there is provided a device for improving flow of air through the nasal cavity of a wearer of the device, the device comprising a substantially U-shaped body forming a bridge for spanning the nostrils of a wearer when the device is in use, first and second bodies receivable in respective ones of the nostrils and interconnected with opposite upright portions of the U-shaped body, each of the first and second bodies comprising a rib which extends outwardly from a portion of the U-shaped body distal to the bridge and ends in a section on which there is provided a relatively large surface area portion, wherein the first and second bodies are each adjustable such that each relatively large surface area portion can be urged against a respective nostril wall, whereby resistance to flow of air is substantially reduced, and wherein each section, and/or the relatively large surface area portion which is provided on it, is configured to hold or carry an agent for release within the nose when the device is in use.

In one embodiment of the invention, the sections are enlarged sections which define the relatively large surface area portions. In another embodiment of the invention, the sections are provided with padding which forms the relatively large surface area portions. In the latter embodiment, the sections may nevertheless be enlarged sections.

In accordance with a third aspect of the present invention, there is provided a dilation device for improving flow of air through a nasal cavity, comprising an elongate U-shaped body having uppermost portions and a lowermost portion, the lowermost portion forming a bridge sufficiently wide to span a nasal septum, and adjustable first and second dilators or bodies mounted to respective ones of the uppermost portions distal to the bridge, each of the first and second bodies or dilators comprising a rib extending outwardly and arcuately from a respective one of the uppermost portions and ending in a section on which there is provided a relatively large surface area portion adapted to abut an internal surface of a nostril, wherein the relatively large surface area portions are defined by padding provided on the sections, and/or wherein the relatively large surface area portions, and/or the sections on which they are provided, are configured to hold or carry an agent for release within the nose when the device is in use.

In accordance with a fourth aspect of the present invention, there is provided a device which can be fitted to a person's nose to facilitate breathing through the nostrils, the device having a frame which comprises a bridge member arranged to span the nostrils when the device is fitted and which further comprises dilator structures interconnected with opposite ends of the bridge member to be received in the nostrils during fitting whereby to be operable to dilate the nostrils, wherein padding is applied to the dilator structures so as to contact internal walls of the nostrils and/or wherein the dilator structures are configured to hold or carry an agent and to release the agent within the nose when the device has been fitted.

Preferably, the frame is resiliently flexible.

Preferably the dilator structures of the frame comprise upwardly convex members.

Preferably, the dilator structures comprise resiliently flexible members. Those members may, in one embodiment of the invention, urge outer walls of the nostrils laterally outwardly under laterally outward biases.

Preferably, said members of the dilator structures comprise ribs.

Preferably, said members of the dilator structures are fitted with large surface area portions which are defined by pads or patches.

In a preferred embodiment of the invention, the padding is arranged to promote comfort and/or grip between the device and nostrils.

The agent may comprise, for example, a fragrance agent, natural product, essential oil, decongestant or a medicated vapour.

In a preferred embodiment of the invention, the device is configured to release a medicament or other agent within the nose. Preferably, the device is configured such that the bodies or dilators carry the medicament or other agent. Preferably, said ribs are configured to hold or carry the medicament. Preferably, each section, and/or said padding with which it is provided, is configured to hold the medicament or other agent for release within the nose.

In one embodiment of the invention, said medicament or other agent is carried, directly or indirectly, on a laterally inner side of each section, for release at positions which are laterally inwardly spaced from the nostril walls. In such an embodiment, the agent may, for example, be one which may be into the nasal passage by inhalation and or one which is inhalable; e.g. it may comprise a mist, vapour or gas.

The advantage of the delivery of a medicament of this type is that a substance comprising the agent can be held away from contact with the skin while allowing inhalation/drawing in of the agent, whereby irritation that can otherwise be caused by direct contact with the skin and/or mucosa can be eliminated.

In another embodiment of the invention, said medicament or other agent is carried, directly or indirectly, on laterally outer sides of the relatively large surface area portions. In such an embodiment, the medicament or other agent may be arranged so as to be absorbed by walls of the nostrils transdermally and/or absorbed by mucosa of said walls.

In a preferred embodiment of the invention, each relatively large surface area portion is defined by a respective pad. Each pad may, for example, be designed to release a said agent, which may comprise a volatile substance, over time.

In the preferred embodiments of the invention, the relatively large surface area portions are adapted for abutting internal wall surfaces of the nostrils of a wearer.

Each of the first and second bodies or dilators can be sized and positioned within a respective nostril cavity to exert a positive pressure against the internal walls of that nostril cavity, thereby opening the cavity to enable passage of air and/or fluid therethrough and to retain the device in its desired position within the nostril.

Preferably, each rib extends arcuately from the respective portion of a U-shaped body defining the bridge, distal to the bridge.

Devices embodying the invention can produce improved air flow capacity for sporting activities and the like.

Each relatively large surface area portion can comprise a flattened section.

Each relatively large surface area portion can comprise a matrix of plastic material containing a saturated load of a drug for controlled delivery of the drug transdermally across the nostril interior walls.

In preferred embodiments of the invention, the device is provided with covers which cover outer surfaces of the relatively large surface area portions and thus preclude contamination of the substance, removal of the covers resulting in the substance becoming exposed such that the agent is released when the device is in use. In one embodiment of the invention, removal of a cover before insertion may expose a substance for transdermal delivery.

The relatively large surface area portion can be provided with or defined by a pad designed to release a volatile substance over time. The pad can be a polymeric matrix including the ointment or methylated topical preparation, and/or a decongestant/essential oils/natural products/amyl nitrite. For extended release, the pad might be various thicknesses. The pad can alternatively act as a depot system for release of an encapsulated solid, which solid can permeate through the walls of the depot at a predetermined rate depending on the thickness if the depot walls, and permeation characteristics of the solid and depot.

The advantage of the delivery of medicated vapour is that the substance can be held away from contact with the skin while allowing inhalation of medicated vapour. Hence the substance is able to function purely as a vapour for inhalation while substantially, eliminating irritation that can otherwise be caused by direct contact with the skin.

In accordance with one preferred feature of the invention, each of the first and second bodies/dilators includes an adjustment mechanism and the ribs comprise members which can be moved laterally by operation of the mechanisms. In accordance with an alternative preferred feature of the invention, each of the first and second bodies/dilators is adjustable by or upon insertion thereof into the respect nostril during fitting of the device. For example, said dilator structures/ribs may be configured to flex resiliently under laterally inward forces imparted to them by laterally outer walls of the nostrils during or upon insertion of the first and second dilators/bodies into the nostrils, whereby each of the first and second dilators/bodies adjusts according to interior dimensions of the respective nostril cavity. In this embodiment of the invention, the dilator structures/ribs may be configured to self-adjust.

In the device in use, each relatively large surface area portion is urged against a nostril wall, whereby resistance to flow of air is substantially reduced.

In a preferred embodiment of the invention, each of the first and second bodies/dilators has a belt adjustable about an attachment mount on an upright portion of a U-shaped body defining the bridge and a said rib extending outwardly and arcuately from a portion of the U-shaped body distal to the bridge, and each said section is connected to a portion of the respective belt. Preferably, the belt and said rib of each of the first and second bodies/dilators form an open channel which defines an air flow pathway within the respective nostril when the device is fitted. Preferably, the each belt is tightenable to allow insertion of the dilator/body into the nostril and thereafter loosenable so as to urge the relatively large surface area portions against walls of the nostril cavities, whereby resistance to flow of air is substantially reduced. Improved air flow capacity for sporting activities and the like may thus result.

In a preferred embodiment of the invention, each of the first and second bodies/dilators has a belt fastened at one end to an upright portion of a U-shaped body defining the bridge, the belt being adjustable by a releaseable locking means mounted on the U-shaped body, the relatively large surface area portion is connected to a portion of the belt, the belt forms a looped structure including the relatively large surface area portion so as to form an opening for ingress of air, and the relatively large surface area portion comprises a matrix or depot comprising a medicament for release in contact with nasal cavity walls.

In a preferred embodiment of the invention, each belt includes a looped strap structure comprising a strap a portion of which is integral with said section, and whereby one end of the strap is fixedly mounted to an upright portion of a U-shaped body, which forms a bridge for spanning the nostrils, and the other end of the strap is adjustable relative to the fixed end, and a releaseable holding means is provided on the upright portion, whereby the holding means includes a fastening means which cooperates with the strap to allow the strap to tighten or loosen, and one end of the strap is adjustably received by the fastening means, and the looped structure is retractable or expandable upon tightening and loosening respectively.

The strap can be adjusted by the holding means to provide a choice of one of a plurality of open structures. The wearer/user can select an appropriate size looped structure relative to the user's respective nostril cavity, and the fastening means can maintain the loop structure at a selected size in a holding condition. Hence the device can be adjusted in situ by a wearer to suit the wearer's specific air flow requirements. In a preferred embodiment of the invention, the holding means is releasably lockable to maintain the loop structure and relatively large surface area portion or enlarged section in a desired position. Owing to the holding means, the size of the looped structure can, advantageously, be maintainable in an adjusted configuration to enable improved airflow that suits a wearer's individual requirements. The holding means can include a security tie member operatively connected thereto which is adapted for fine adjustment of the looped structure in situ. Hence the amount of adjustability of the device can be controlled by the wearer.

A substantial benefit a reversible lockability of each holding means is its use in changing the dimensions of the respective dilator/body when the user has a different condition and the nostril cavity size has changed, or where a user over-expands the device prior to insertion; the holding means may allow for further fine adjustment. Such a condition can occur due to inflamed sinuses or because of colds, influenza and other nose-affecting ailments or due to physical damage such as sunburnt noses, broken or damaged noses.

The holding means can comprise mating or interlocking components on the upright portion of the U-shaped body and on one end of the strap to releasably lock the strap.

In one embodiment, the holding means can include a sleeve or housing having a series of internal teeth which resiliently and releasably engage with one end of the strap to reduce or enlarge the channel opening in the looped structure.

The rib members can be configured to move laterally to expand or contract the looped opening channel formed by the strap structure by passing one end of the strap through the sleeve until a tooth in the sleeve engages an appropriate end section of the strap.

In one embodiment of the invention, a medicament or agent delivery system can be mounted on a U-shaped body defining the bridge of the device.

The device in use may improve flow of air through the nasal cavity of a wearer during activity such as cycling or running or like exercise, substantially minimising air flow resistance.

The device according to preferred embodiments of the invention is lightweight and provides less resistance to air flow than prior art devices. As a result, it is particularly well suited for use during sporting activities such as cycling and the like which require greater intake of air, hence reducing the sense of obstruction felt when exhaling through the nose under duress.

The device according to particular preferred embodiments of the invention is suited to any sized nostril, is economic, reusable and aesthetically pleasing. The device can be made from polymeric materials, and can deliver any of a range of agents including fragrance, natural products, essential oils, decongestants or medicated vapour.

The device according to particular preferred embodiments of the invention includes a means for delivery of a medicament or medicated vapour. In one embodiment, the relatively large surface area portions or enlarged sections comprise a matrix of plastic material containing a saturated load of a drug for controlled delivery of the drug transdermally across the nasal cavity walls.

Each relatively large surface area portion or enlarged section can include a depot or reservoir for housing a medicament. The reservoir or depot can be defined by a pitted surface of the relatively large surface area portion, which in contact with the nasal mucosa releases an effective amount of an active agent. In storage, the relatively large surface area portion can be covered with a removable adhesive.

Alternatively, each relatively large surface area portion can include one or more recesses—e.g. a series of spaced apart parallel grooves—into which an agent/medicament formulation can be deposited. When the relatively large surface area portion contacts internal nasal cavity walls, the recesse(s)/grooves may substantially minimise direct contact between the agent/formulation and nostril cavity wall surfaces. This is particularly useful where the agent/formulation releases a medicated vapour.

In a further embodiment of the invention, each relatively large surface area portion and/or section includes a matrix suitable for release of volatile substances including amyl nitrite to treat heart disease such as angina and also to treat cyanide poisoning.

One form of matrix can be heterogeneous including a polymer throughout which there is dispersed a medicament, and wherein the matrix is partially sealed so as to define an air flow pathway such that incoming air flow initiates release of volatile agent within the polymer matrix.

The mode of delivery can include any of the following: (1) the “active agent” could be infused through the relatively surface area portions or said sections by infusing drug into a matrix; (2) the “substance” could be deposited in dimples that contact the drug directly with the inside of the nose or (3) the “substance” could be placed in a patch (pad) which sticks to or defines the relatively large surface area portion, or which is applied to each said section.

In a further embodiment of the invention, each of the relatively large surface area portions and/or said sections can include or be provided with a matrix suitable for release of volatile substances including amyl nitrite to treat heart disease such, as angina and also to treat cyanide poisoning. One form of matrix can be heterogeneous including a polymer throughout which there is dispersed a medicament, and wherein the matrix is partially sealed so as to define an air flow pathway such that incoming air flow initiates release of volatile agent within the polymer matrix.

The relatively large surface area portion or said section can be provided with or defined by a pad designed to release a volatile substance over time. The pad can be a polymeric matrix including essential oils/natural products/amyl nitrite. For extended release, the pad might be various thicknesses. The pad can alternatively act as a depot system for release of an encapsulated solid, which solid can permeate through the walls of the depot at a predetermined rate depending on the thickness if the depot walls, and permeation characteristics of the solid and depot.

An advantage of the delivery of medicated vapour by devices according to particular preferred embodiments of the invention is that any substance can be held away from contact with the skin while allowing inhalation of medicated vapour. Hence the substance is able to function purely as a vapour for inhalation while substantially eliminating irritation that can otherwise be caused by direct contact with the skin. In one instance a medicated delivery system can be mounted, any one or more of the bodies.

The flattened sections, relatively large surface area portions or enlarged sections may abut against internal nasal cavity walls to improve a wearer's level of comfort, and may terminate on a portion of the belt or strap (if present).

In a preferred embodiment of the invention, each of said upright portions is configured with an enlarged section provided with padding which forms a relatively large surface area portion arranged to contact the septum within the respective nostril. This padding may improve comfort and/or grip between the device and the nose when the device is worn. This padding may be made of and/or have the configuration of the padding outlined above in respect of the enlarged sections.

In a preferred embodiment of the invention, each of said upright portions is configured with an enlarged section which is configured to hold, directly or indirectly, an agent for release within the nose.

According to a fifth aspect of the present invention, there is provided said device fitted to a person's nose, in use. In one embodiment of the invention, the device carries or holds said agent whereby to release it within the nose.

Further exemplifications of the present invention will be described as follows with reference to the accompanying drawings. A brief description of the drawings follows:

### BRIEF DESCRIPTION OF THE DRAWINGS

The invention will be described, by way of non-limiting example only, with reference to the accompanying drawings, in which:

FIGS. 1A, 1B, 1C, ID and 1E are plan, front elevation, cross-sectional (A-A), perspective and side elevation views, respectively, of a device for facilitating breathing through the nose in accordance with a first embodiment of the present invention;

FIGS. 2A, 2B, 2C and 2D are plan, front elevation, perspective and side elevation views, respectively, of a device for facilitating breathing through the nose in accordance with a second embodiment of the present invention;

FIGS. 3A, 3B and 3C are perspective views of a device for facilitating breathing through the nose in accordance with a third embodiment of the present invention in which dilators of the device are shown in fully expanded, fully contracted and disassembled conditions, respectively;

FIGS. 3D and 3E are front elevation views of the device of the third embodiment showing dimensions of the device when dilators thereof are in fully contracted and fully expanded conditions, respectively;

FIG. 3F is a perspective view of the device of the third embodiment showing dimensions of a bridge thereof;

FIGS. 4A and 4B are perspective views of a device for facilitating breathing through the nose in accordance with a fourth embodiment of the present invention in which dilators of the device are shown in fully contracted and fully expanded conditions, respectively; and

FIGS. 5A and 5B are perspective and front elevation views, respectively, of of a device for facilitating breathing through the nose in accordance with a fifth embodiment of the present invention, in which dilators of the device are shown in fully expanded conditions.

### DETAILED DESCRIPTION OF THE PREFERRED EMBODIMENTS

The content of the specification and drawings of international patent application no. PCT/AU2012/000898 as originally filed is incorporated herein by reference:

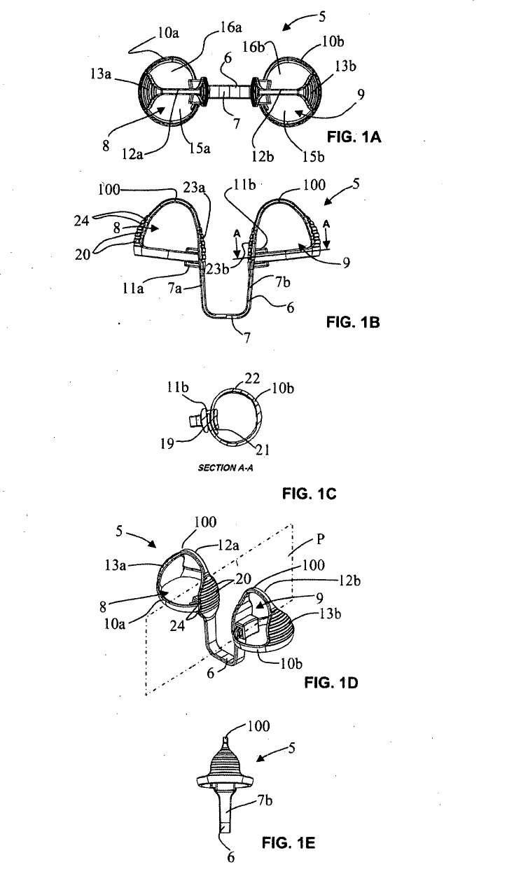

Shown in FIGS. 1A to 1E is an improved adjustable nostril dilation device 5 in accordance with a first preferred embodiment of the invention. The device can be fitted to a person's nose to improve airflow through the nostrils and thus breathing through the nasal cavity, e.g. while the person exercises.

The dilation device 5 is moulded as a single piece from resiliently flexible polymeric material and includes a U-shaped body 6 defining a bridge 7 and generally upright support elements 7a and 7b which are separated by the bridge 7. When the device is fitted, the bridge 7 extends across the exterior of the columella (the fleshy external end of the nasal septum) and thus spans the openings into the adjacent nostril cavities of the wearer. The support elements 7a and 7b are upwardly divergent, to a slight extent, about a central upright plane P, whereby to facilitate fitting of the device 5. The device 5 is configured symmetrically about the plane P.

The device 5 further includes first 8 and second 9 adjustable bodies or dilators interconnected with opposite upright portions 7a and 7b respectively of the U-shaped body 6.

The first and second bodies 8, 9 include belts 10a and 10b, respectively, each of which is wound into a loop. The belts 10a and 10b are received through collars 11a and 11b provided on upright portions 7a and 7b respectively, and adjustable about those collars, as will be described in further detail later. The first and second bodies 8, 9 also include ribs 12a and 12b, respectively, each of which is bent, in a substantially upright plane which is substantially perpendicular to the central plane P, so as to have an arcuate and upwardly convex configuration.

As can be seen clearly in FIG. 1D, the rib 12a includes a narrow portion 100 interconnected with and dependent from the upper end portion of the upright support element 7a, and ends in an enlarged section defining a relatively large surface area portion 13a which is interconnected with the belt 10a. Similarly, the rib 12b includes a narrow portion 100 interconnected with and dependent from the upper end portion of the upright support element 7b, and ends in an enlarged section defining a relatively large surface area portion 13b which is interconnected with the belt 10b. The bodies 8, 9 are configured such that, when the device is fitted to a person's nose, the portions 13a and 13b contact the interior faces of laterally outer walls of the person's nostrils, urging those walls laterally outwardly to dilate the nostrils. Owing to their having surface areas which are large compared to those of the portions 100, the portions 13a, 13b distribute compressive loads exerted on the laterally outer walls of the nostrils, thereby mitigating or eliminating irritation and enhancing comfort.

In the body 8, the belt 10a and rib 12a form front 15a and rear 16a substantially circular or elliptical openings. Similarly, in the body 9, the belt 10b and rib 12b form front 15b and rear 16b substantially circular or elliptical openings. When the device 5 is fitted, air can flow through the openings 15a, 15b, 16a, 16b substantially free of obstruction as the person breathes through his or her nose.

Referring to FIG. 1C, which is a view of section A-A through the body 9 labeled in FIG. 1B and a mirror image of the corresponding section through the body 8, each of the belts 10a, 10b is permanently fixed, at one end portion 19 thereof, to the U-shaped body 6. In the example shown, each, end portion 19 is positioned within the respective collar and attached to the respective upright element defining the laterally inner wall of the collar, though in other examples that portion may be positioned and attached elsewhere without departure from the invention. The opposite end portion 21 of each belt is passed through the respective collar and releasably held therein. Each end portion 21 is configured with formations (not shown) arranged therealong to be interlockingly engageable with corresponding formations (also not shown) within the respective collar. The formations and corresponding formations may, for example, comprise intermeshable teeth, and be formed either on the radially inner face of the end portion 21 and the radially outer face of the laterally outer wall of the collar respectively, or on the radially outer face of end portion and the radially inner face of the belt end portion 19 respectively.

The interengaging formations and corresponding formations in each of the bodies 8 and 9 may be configured such that, when each collar is in a substantially undeformed condition, they permit the respective end portion 21 to be moved through the collar only in one direction, which is that in which the free end of the end portion 21 faces (e.g., in a manner generally consistent with interengaging formations in a cable tie), and thus allow only for the belt to be more tightly wound and thereby reduced in circumference, e.g. via pressing of front and rear sections of the respective belt between finger and thumb, whereby the bodies 8 and 9 comprise adjustment mechanisms which operate on a ratchet principle. Alternatively, resilient deformability of the collar and/or the end portion 21 may allow for the end portion 21 to be moved through the collar additionally in the opposite direction, whereby the bodies 8 and 9 comprise adjustment mechanisms which are not ratchet mechanisms as such. An inherent resilience of each belt may impart to the respective loop 22 formed thereby a radially outward bias the strength of which increases as the circumference of the loop 22 decreases. Also as that circumference decreases, the laterally outer ends of the respective rib is drawn, by the belt, closer to the U-shaped body, whereby the degree of bending of the rib progressively increases. An inherent resilience of each rib may also impart to the respective loop radially outward bias the strength of which increases as the degree to which the rib is bent increases.

When the belts are wound/tightened (in the manner just described) to an extent such that their circumferences are sufficiently small, the bodies 8 and 9 can be inserted into the respective nostrils.

It may be that disengagement of the interengaged formations is thereafter effected, e.g. by pinching the collar or a part of the device 5 adjacent the collar, so as to place the collar into a deformed condition, whereby either or each of the aforementioned biases forces the respective belt to loosen and loop 22 to expand, urging the relatively large surface area portion laterally outwardly against the inner face of the nostril laterally outer wall and thus dilating the nostril. The collars may, to facilitate the deformation thereof, be formed from material which is soft compared to that from which the U-shaped body 6 is formed. Instead, the device 5 may include actuators (not shown) which are operable by the wearer, after the bodies 8 and 9 have been inserted into the nostrils, to effect the disengagement. When the nostril cavity is expanded sufficiently, the teeth can be interlocked by the releaseable holding means to retain the ribs in a new/expanded condition.

Alternatively, it may be that the resistance, offered by the interengagement between the collar and belt in each of the bodies' adjustment mechanisms, to expansion of the respective loop 22 is thereafter forcedly overcome, e.g. by the wearer forcing front and rear sections of the respective belt outwardly using digits on his or her hand(s), perhaps with some assistance from either or each of the aforementioned biases, to effect the loosening of the respective belt and expansion of the loop 22.

Each narrow portion 100 acts as a living hinge allowing hinged movement of the respective enlarged surface area portion when the respective loop circumference is adjusted. When each of the enlarged surface area portions 13a, 13b is hingedly displaced between a retracted condition and an expanded condition, via loosening of the respective belt, that portion exerts a positive pressure against the interior surface of the laterally outer wall of the respective nostril. Owing to the narrowness of the portions 100, air flow past those portions is substantially unobstructed thereby.

The device 5 can remain fitted without being vulnerable to inadvertent dislodgement, each of the ribs 12a, 12b being maintained at a selected degree of divergence by interengagement of the aforementioned formations, with the portions 13a and 13b bracing against the laterally outer walls of the nostrils to assist in holding the device 5 in place.

The device 5 can be manually adjusted in situ to control the degree of nostril dilation. The upright support members 7a, 7b are received against opposite sides of the septum. As can best be seen in FIG. 1D, members 7a and 7b include, at upper ends thereof, enlarged or thickened sections which define relatively large surface area portions 23a and 23b, which are arranged to contact the septum when the device 5 is fitted, distributing compressive loads exerted on the septum as a result of laterally outward flexure of the members 7a and 7b as they are fitted over the septum, thereby mitigating or eliminating irritation and enhancing comfort.

The U-shaped body 6, or at least the bridge portion 7 thereof, may be substantially colourless and transparent or translucent, or alternatively flesh-coloured, so as to render the device as inconspicuous as possible. Alternatively, it may be coloured in a manner so as to be conspicuous.

Each of the portions 13a, 13b, 23a, 23b is configured with outer surface formations, which in the present embodiment comprise raised portions, in the form of ribs 20, and recesses, in the form of grooves 24, which are arranged in alternating fashion. The ribs 20 and grooves 24 in this embodiment are substantially parallel but in other embodiments need not be. The ribs 24 and grooves 20 in this embodiment extend in forwards-backwards directions but in other embodiments need not do. In other embodiments, the formations may, for example, comprise, instead of grooves, recesses in the form of pits, which could be arranged in an array over the respective outer surface. The formations on each of the portions 13a, 13b, 23a, 23b, advantageously, enhance grip between that portion and the interior nostril face against which it is received and/or anchors or holds a substance which comprises an agent releasable into the nose when the device 5 is in use (and may thereby define a reservoir or depot which holds the substance). The agent may comprise, for example, a medicament and/or a fragrancing agent. The substance may be one that can be applied, and/or re-applied, by the user of the device 5, e.g. by wiping, smearing or painting of the substance on the outer surface(s) of any one or more of the enlarged portions, or one which has been pre-applied to the device 5 as at the time of supply.

The substance may be distributed over substantially the entirety of the outer surfaces of the portions 13a and 13b and/or portions 23a and 23b when applied, or may be applied such that it occupies only the recesses 20 (e.g., substance may be wiped or scraped off the portions, after application, such that the substance which remains is substantially confined to the recesses 20). The substance may, particularly in the latter situation, beheld substantially free of direct contact with the skin inside the nostrils when the device 5 is in use though in contact with nasal mucosa inside the nostrils, whereby to release an effective amount of the agent into the mucosa. Alternatively or additionally, the substance may be applied to the laterally inward facing (opposite) surfaces of the enlarged sections 13a, 13b and/or the laterally inward facing (opposite) surfaces of the enlarged sections 23a, 23b, whereby to be held clear of contact with interior walls of the nostrils; as may be desirable particularly where the substance releases a gas, vapour or mist. Alternatively, the substance may lie in contact with, or sufficiently close to, the skin such that the agent is absorbed transdermally.

Alternatively or additionally, the substance may be one which releases the agent as a vapour or gas and may so release the agent for inhalation by the person using the device. In this situation, the agent may comprise, for example, a menthol preparation, and may be held substantially free of contact with the both skin and mucosa in the nostrils, such that irritation which would be caused by such contact is eliminated, and be released within the nose whereby to be inhaled.

The device 5 may be provided with removable, e.g. peel-off, covers which cover the outer surfaces of the enlarged portions to which the substance is applied, and thus preclude contamination of the substance, removal of the covers resulting in the substance becoming exposed such that the agent is released when the device 5 is in use.

In another embodiment, the portions 13a and 13b and/or the portions 23a and 23b may each be provided with a matrix of plastic material containing a saturated load of the substance for controlled delivery thereof transdermally.

Application of the substance to the device 5 can comprise, for example, any one of: (a) infusion of the substance into the enlarged surface area portions 13a, 13b and/or the portions 23a and 23b, through matrices with which those portions are provided; (b) deposition of the substance in dimples (not shown) arranged to contact the substance directly with the inner surfaces of the nose; and (c) application of a patch or pad to each of the portions 13a, 13b and/or the portions 23a, 23b, which patch or pad may, for example, be one which is applied by sticking it to the portion or overmoulding it onto the portion.

Each relatively large surface area portion 13a, 13b can, without departure from the invention, be defined by a pad designed to release, over time, a volatile substance, one or more essential oils, one or more natural products or amyl nitrite. For extended release, the pads might be provided in varying thicknesses.

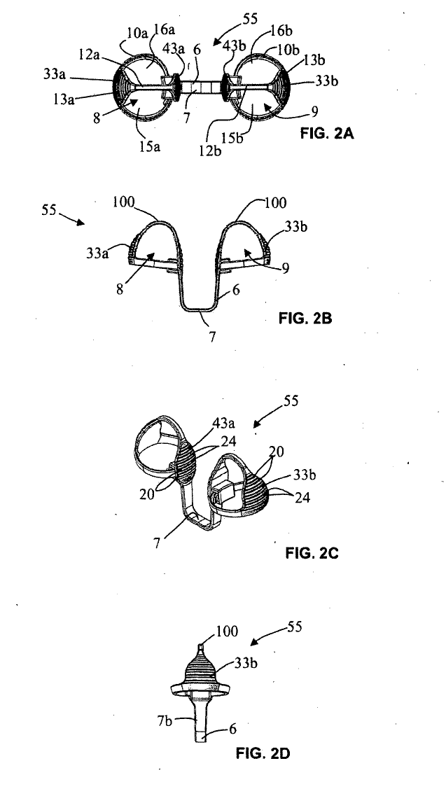

Shown in FIGS. 2A to 2D is an improved adjustable nostril dilation device 55 in accordance with a second preferred embodiment of the invention. The same reference numerals as have been used in respect of the first embodiment will be used to denote and refer to the same or corresponding features of the second embodiment. The device 55 is the same as the device 5 except that the ribs 12a and 12b, instead of comprising enlarged sections which themselves define the relatively large surface area portions 13a, 13b, comprise enlarged sections onto which are fixed relatively soft material portions forming pads 33a and 33b which define the relatively large surface area portions 13a and 13b respectively, and that the support members 7a and 7b, instead of comprising enlarged or thickened sections which themselves define the relatively large surface area portions 23a, 23b, comprise enlarged or thickened sections onto which are fixed relatively soft material portions forming pads 43a and 43b which define the relatively large surface area portions 23a and 23b respectively. The soft material, although preferably medical grade, may comprise any one of several elastomers, including natural and synthetic elastomers, silicone elastomers, latex, isoprenes, polyisoprenes, polystyrene butadienes, nitrile elastomers, butyl elastomers, ethylene propylene terpolymers, silicone elastomers, neoprenes, polysulfide, poly acetyl, eperchloride, fluoroelastomers, hypalon, halogenated butyl, polyurethanes and thermoplastic elastomers, such as those marketed under the names Mediprene and Thermolast-M. The pads 33a, 33b, 43a, 43b are preferably overmoulded to the respective enlarged sections but may, alternatively, be fixed to those sections in a different manner—e.g. via adhesive. The pads 33a, 33b, 43a, 43b, because they are relatively soft, afford the device 55 improved comfort characteristics and/or promote gripping between the portions 13a, 13b, 23a, 23b and the interior faces of the nostrils which they contact. The device 55 may, like device 5, be provided with removable, e.g. peel-off, covers which cover the outer surfaces of the relatively large surface area portions 13a, 13b and/or 23a, 23b in versions of the device 55 where the substance is applied thereto, and thus preclude contamination of the substance, removal of the covers resulting in the substance becoming exposed such that the agent is released when the device 55 is in use.

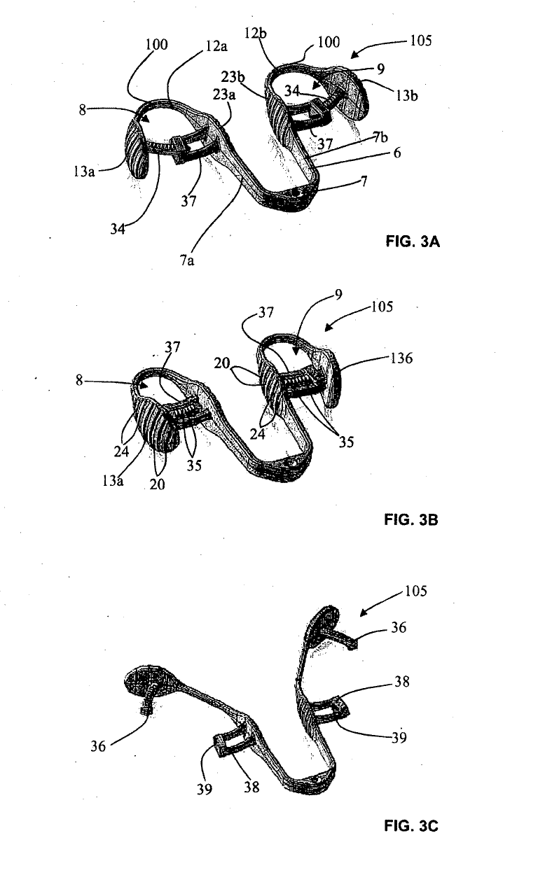

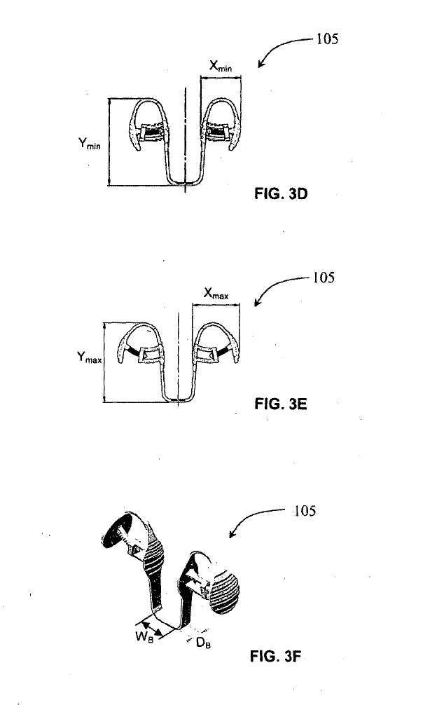

Shown in FIGS. 3A to 3F is an improved adjustable nostril dilation device 105 in accordance with a third preferred embodiment of the invention. The same reference numerals as have been used in respect of the first and second embodiments will be used to denote and refer to the same or corresponding features of the third embodiment. The device 105 is substantially the same as the device 5, except with respect to the adjustment mechanisms and the shapes of the enlarged sections which define the relatively large surface area portions 13a, 13b.

The adjustment mechanism of each of the bodies/dilators 8 and 9 in the device 105, instead of comprising a wound belt and a collar which receives the belt, comprises an elongate member 34 which projects arcuately and transversely from an inner side of the respective one of the enlarged surface area portions 13a and 13b and is configured with axially spaced apart teeth 35 on both sides thereof, and a bracket 37 which projects transversely from an outer side of the enlarged section 23a/23b, the bracket 37 being configured at its free with a boss 38 formed with an aperture 39 (see FIG. 3C) into which the elongate member 34 is received. The teeth 35 and boss 38/aperture 39 are configured such that successive pairs of adjacent teeth 35 along the length of the member 34 engage the boss 38 at either end of the aperture 39 to hold the portion 13a/13b fixed with respect to the adjacent portion 23a/23b at respective lateral spacings from the latter portion. The lateral spacing between the portion 23a and the adjacent portion 13a can be adjusted by manually pushing those portions together or pulling them apart with sufficient force that the resilient deformability of the respective boss 38 and/or teeth 35 permits the latter to be forced through the former.

Similarly, the lateral spacing between the portion 23b and the adjacent portion 13b can be adjusted by manually pushing those portions together or pulling them apart with sufficient force that the resilient deformability of the respective boss 38 and/or teeth 35 permits the latter to be forced through the former. The dilators 8 and 9 may thus be caused to assume contracted configurations, such as those shown in FIGS. 3B, 3D and 3E, for receipt into the nostrils (during fitting of the device 105), and thereafter caused to assume any one of plural expanded conditions, a maximum one of which is shown in FIGS. 3A and 3F, the expanded condition being selected according to the anatomy of the nostrils and/or required degree of dilation thereof.

Each of the members 34 is configured at its free end with a head 36 that is larger cross-sectionally than the aperture 39 to an extent sufficient that precludes the member 34 from being withdrawn from the boss 38 unless a relatively large pulling force is applied to the member 34. Each of the heads 36 is forwardly convergent to facilitate insertion of the member 34 into the boss 38 through the aperture 39, e.g. when the device 105 is being taken out of a disassembled condition as shown in FIG. 3C and placed into an assembled condition as shown in FIGS. 3A, 3B and 3D to 3F.

An inherent resilience of each of the ribs 12a and 12b may impart to the respective relatively large surface area portion 13a/13b an outward bias, the strength of which may increase as the degree to which the rib is bent (from the condition shown in FIG. 3C) increases, which bias may assist in retention of the respective dilator 8/9 in the expanded condition in use.

In the device 105, the relatively surface area portions 13a, 13b have lower ends which, instead of being straight (as in the devices 5 and 55), are downwardly convex.

Referring to FIG. 3D, when either of the dilators 8 and 9 is in a fully contracted condition, the lateral spacing Xₘᵢₙ between the outermost extents of the relatively large surface area portion 13a or 13b and the relatively large surface area portion 23a or 23b is about 10 mm, and the difference in level between the lowermost extent of the bridge 7 and the uppermost extent of the rib 12a or 12b Yₘᵢₙ is about 22 mm. Referring to FIG. 3E, when either of the dilators 8 and 9 is in a fully expanded condition, the lateral spacing Xₘₐₓ between the outermost extents of the relatively large surface area portion 13a or 13b and the relatively large surface area portion 23a or 23b is about 13.5 mm, and the difference in level between the lowermost extent of the bridge 7 and the uppermost extent of the rib 12a or 12b Yₘₐₓ is about 22.5 mm. Referring to FIG. 3E, the bridge 7 has a width of W_{B} of about 7.5 mm and a depth D_{B} of about 2.5 mm. The devices of the other embodiments described and illustrated herein may have similar dimensions.

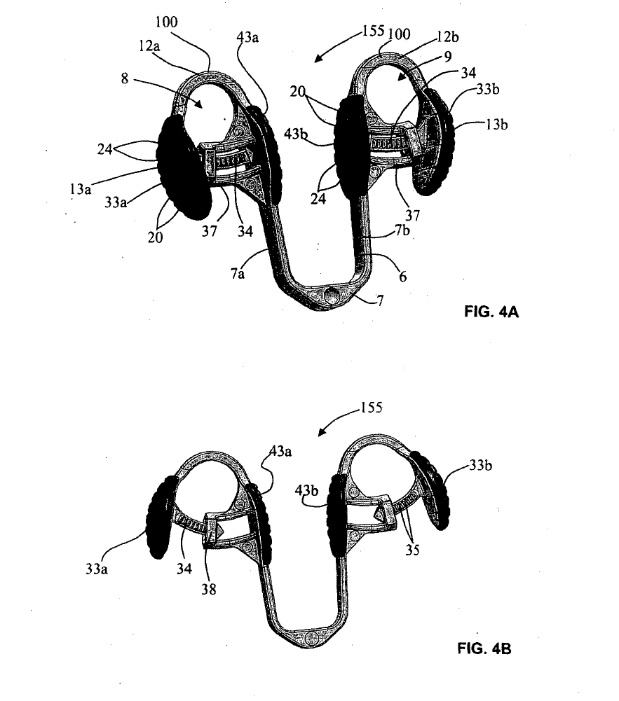

Shown in FIGS. 4A and 4B is an improved adjustable nostril dilation device 155 in accordance with a fourth preferred embodiment of the invention. The same reference numerals as have been used in respect of the first, second and third embodiments will be used to denote and refer to the same or corresponding features of the fourth embodiment. The device 155 is the same as the device 105 except that the ribs 12a and 12b, instead of comprising enlarged sections which themselves define the relatively large surface area portions 13a, 13b, comprise enlarged sections onto which are fixed relatively soft material portions forming pads 33a and 33b which define the relatively large surface area portions 13a and 13b respectively, and that the support members 7a and 7b, instead of comprising enlarged or thickened sections which themselves define the relatively large surface area portions 23a, 23b, comprise enlarged or thickened sections onto which are fixed relatively soft material portions forming pads 43a and 43b which define the relatively large surface area portions 23a and 23b respectively. The soft material may, for example, comprise any one of the materials outlined above in the description of the device 55. The pads 33a, 33b, 43a, 43b are preferably overmoulded to the respective enlarged sections but may, alternatively, be fixed to those sections in a different manner—e.g. via adhesive. The pads 33a, 33b, 43a, 43b, because they are relatively soft, afford the device 155 improved comfort characteristics and/or promote gripping between the portions 13a, 13b, 23a, 23b and the interior faces of the nostrils which they contact. The device 155 may, like device 105, be provided with removable, e.g. peel-off, covers which cover the outer surfaces of the relatively large surface area portions 13a, 13b and/or 23a, 23b in versions of the device 155 where the substance is applied thereto, and thus preclude contamination of the substance, removal of the covers resulting in the substance becoming exposed such that the agent is released when the device 55 is in use.

The dilators/bodies 8 and 9 in each of the devices 5, 55, 105 and 155 can, advantageously, be adjusted to user-selected degrees of expansion when the device is fitted.

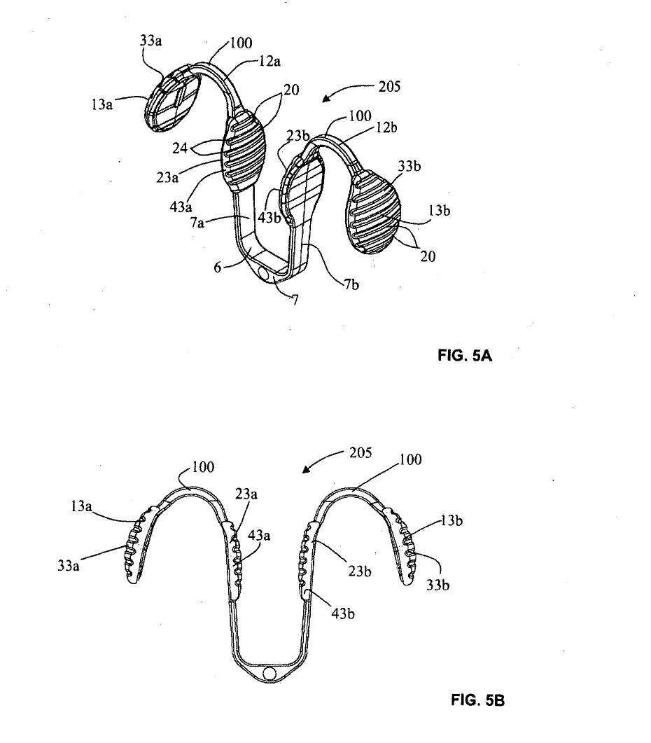

Shown in FIGS. 5A and 5B is an improved adjustable nostril dilation device 205 in accordance with a fifth preferred embodiment of the invention. The same reference numerals as have been used in respect of the first, second, third and fourth embodiments will be used to denote and refer to the same or corresponding features of the fifth embodiment. The device 205 is substantially the same as the device 155 except that the elongate members 34 and brackets 37 are omitted whereby the relatively large surface area portions 13a and 13b must, after being pushed laterally inwardly into positions in which they are close to the portions 23a, 23b, such that the dilators/bodies 8, 9 assume contracted conditions, be manually held in order to remain in those positions, and whereby the portions 13a and 13b will, once the bodies/dilators 8, 9 have been inserted into the nostrils, assume a lateral spacing from the portions 23a and 23b according to the stiffness of the ribs 12a and 12b and the anatomy of the nostrils, the bodies/dilators 8, 9 thus being self-adjustable in accordance with the stiffness and anatomy.

Without departure from the invention, any one of the devices 5, 55, 105, 155 and 205 may be modified such that it is, alternatively or additionally, configured such that a said agent is held, either directly or indirectly, by the laterally inner sides of the enlarged sections 13a, 13b, and/or the laterally outer sides of the enlarged sections 23a, 23b—e.g. elements, such as pads, patches or gel elements, may be applied to said sides. The agent may be one which is volatile and/or is inhalable and may, owing to its being held at these locations, be clear of tissue surfaces/mucosa inside the nostrils.

Each of the devices 5, 55, 105, 155 and 205 is, advantageously, relatively simple to produce and to fit, configured to hold reliably and comfortably to the nose, relatively inconspicuous in use and able to hold and release an agent into the nose, which may benefit the wearer. Each device is particularly suitable, though by no means solely intended, for use when the wearer thereof is exercising.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A device for improving flow of air through the nasal cavity of a wearer of the device, the device comprising:
a substantially U-shaped body forming a bridge for spanning a septum of a nose of the wearer when the device is in use; and
a first body and a second body interconnected with opposite upright portions of the U-shaped body, each comprising:
a rib which extends outwardly from a portion of the U-shaped body distal to the bridge and ends in a section provided with padding which forms a relatively large surface area portion; and
wherein a recess is provided in the section and is arranged to receive a medicament or agent,
wherein the first and second bodies are adjustable such that each relatively large surface area portion can be urged against a nostril wall, whereby resistance to flow of air is substantially reduced.

2. The device for improving flow of air through the nasal cavity of a wearer of the device according to claim 1, the device comprising:
a substantially U-shaped body forming a bridge for spanning a septum of a nose of the wearer when the device is in use,
first and second bodies receivable in respective ones of the nostrils and interconnected with opposite upright portions of the U shaped body, each of the first and second bodies comprising:
a rib which extends outwardly from a portion of the U-shaped body distal to the bridge and ends in a section on which there is provided a relatively large surface area portion,
wherein at least one recess is provided in the section and is arranged to receive an agent,
wherein the first and second bodies are each adjustable such that each relatively large surface area portion can be urged against a respective nostril wall, whereby resistance to flow of air is substantially reduced.

3. The device according to claim 2, wherein the sections are enlarged sections which define the relatively large surface area portions.

4. The device according to claim 2, wherein the sections are provided with padding which forms the relatively large surface area portions.

5. A dilation device for improving flow of air through a nasal cavity, comprising:
an elongate U-shaped body having uppermost portions and a lowermost portion, the lowermost portion forming a bridge sufficiently wide to span a nasal septum, and
adjustable first and second dilators or bodies mounted to respective ones of the uppermost portions distal to the bridge, each of the first and second bodies or dilators comprising:
a rib extending outwardly and arcuately from a respective one of the uppermost portions and ending in a section on which there is provided a relatively large surface area portion adapted to abut an internal surface of a nostril,
wherein at least one recess is provided in the section and is arranged to receive a medicament or agent, and
wherein the relatively large surface area portions are defined by padding provided on the sections.

6. The device which can be fitted to a person's nose to facilitate breathing through the nostrils, the device having:
a frame which comprises a bridge member arranged to span the nostrils when the device is fitted, and
dilator structures interconnected with opposite ends of the bridge member to be received in the nostrils during fitting whereby to be operable to dilate the nostrils,
wherein padding is applied to the dilator structures so as to contact internal walls of the nostrils and wherein at least one recess is provided in the dilator structures and is arranged to receive an agent and to release the agent within the nose when the device has been fitted.

7. The device according to claim 1, wherein the recess is a depot.

8. The device according to claim 1, wherein the recess is a reservoir.

9. The device according to claim 1, fitted to a person's nose, in use.

10. The device according to claim 5, wherein the recess is a depot.

11. The device according to claim 5, wherein the recess is a reservoir.

12. The device according to claim 5, fitted to a person's nose, in use

13. The device according to claim 6, wherein the recess is a depot.

14. The device according to claim 6, wherein the recess is a reservoir.

15. The device according to claim 6, fitted to a person's nose, in use.
